(19) United States
(12) Patent Application Publication
(10) Pub. No.: US 2015/0139957 A1
(43) Pub. Date: May 21, 2015

(54) GROWTH ENHANCING ADDITIVE FROM POMEGRANATE PLANT FOR POULTRY FEED

(71) Applicants: GANIR (1992) LTD., M.P. Sde Gat (IL); MILOUBAR, CO-OP FOR AGRICLUTURAL PRODUCE LTD., Mobile Post Oshrat (IL)

(72) Inventors: Yuval Katzir, M.P. Hefer (IL); Eli Budman, M.P. Hof Carmel (IL); Arie Yaari, Nahariya (IL); Eliezer Hendel, M.P. Western Galilee (IL)

(73) Assignee: GANIR (1992) LTD, M.P.Sde Gat (IL)

(21) Appl. No.: 14/401,382

(22) PCT Filed: May 12, 2013
(86) PCT No.: PCT/IL2013/050412

§ 371 Date: Nov. 14, 2014

(30) Foreign Application Priority Data
May 15, 2012 (IL) 219810

(51) Int. Cl. A61K 36/185; A23K 1/18; A23K 1/16; A61K 45/06

(52) U.S. Cl. CPC A61K 36/185; A61K 45/06; A23K 1/1826; A23K 1/1646
USPC 424/93.4; 424/769; 424/94.1; 424/94.6

(57) ABSTRACT

A method for enhancing the growth, increasing feed utilization and reducing mortality in poultry (such as chickens and turkeys) by adding a pomegranate-derived additive to their feed. Pomegranate-derived additive and its use for enhancing growth of poultry without growth-promoting antibiotics.

### FIELD OF THE INVENTION

The present invention relates to an additive for poultry feed, for use in enhancing the animal health and growth, and particularly in substituting the growth promoting antibiotics in the poultry nutrition.

### BACKGROUND OF THE INVENTION

The routine addition of antibiotics to animal fodder largely increased the productivity of livestock industry, as many microbial infections were thus prevented, and as the general health of animals was enhanced. More than three quarters of antibiotics produced in the U.S. have been used for this purpose. The negative effects of such overuse include environmental pollution by foreign chemicals, and appearance of drug-resistant microbial strains. The increased environmental awareness of the public, combined with stronger concerns for animals' well being, has resulted in searching for antibiotic substitutes which would ensure efficient and healthy development of the farm animals. For example in Europe, the use of antibiotics as growth promoter in poultry feed was entirely forbidden. Alternative means for replacing the antibiotics in fodder have been examined, including enzymes, prebiotics, probiotics, phytobiotics, and acids [Castanon J. I. R.: Poult Sci. 86 (2007) 2466-71]. However, no universal solution has been found, and the poultry industry faces additional setbacks, including new rules about poultry housing, ban on use of bone and meat in feed, and other new regulations. The negative effect of the antibiotics ban in poultry feed, together with the new housing and nutrition requirements pose hard challenges to the poultry farming, particularly in the time of increasing demand for poultry meat.

It is therefore an object of this invention to provide an additive for poultry feed to replace antibiotics.

It is another object of this invention to provide a non-antibiotic feed additive for poultry industry enabling to improve at least one of the parameters selected from body weight, feed utilization, and fowl mortality.

It is still another object of this invention to provide a poultry feed additive which would lower the contamination of the environment with foreign and harmful chemicals.

It is a further object of this invention to provide a poultry feed additive which would enhance the production of safe and high-quality poultry meat.

Other objects and advantages of present invention will appear as description proceeds.

### SUMMARY OF THE INVENTION

The present invention provides a method for enhancing the growth of poultry, comprising admixing to the feed for said poultry an additive derived from pomegranate plant. Said poultry preferably comprise chickens for fattening and turkeys, such as broilers. Said additive preferably substitutes for growth-promoting antibiotics, The invention aims at a method for enhancing the growth of poultry, comprising admixing to the feed for said poultry an additive derived from pomegranate plant, and further comprising omitting growth-promoting antibiotics in their feed. Said additive is prepared by processing a pomegranate part selected from the group consisting of leaves, flowers, fruits, fruit cores, fruit peels, fruit pulp membranes, fruit arils, and mixtures thereof. Said additive may be a dried pomegranate part, an extract, an extract concentrate in liquid or powder form, or a mixture thereof. In a preferred embodiment of the invention, the method for enhancing the growth of poultry, comprises adding to the feed for said poultry an additive comprising pomegranate peels selected from the group consisting of dried pomegranate peels, an extract from pomegranate peels, and silage of pomegranate peels. The method may comprise enhancing the growth of poultry by adding to the feed for said poultry a milled pomegranate part or an extract of a pomegranate part. Said additive is preferably added to said feed in an amount of from about 0.01 kg to about 50 kg per ton feed, for example between 0.1 kg and 10 kg per one ton feed. Said additive may be a solid or a liquid, and may further comprise a component selected from taste mask, sweetener, and tannin neutralizing component. The growth enhancement in a method according to the invention may result in increased average weight of said poultry, or in increased efficiency of the feed utilization, or in lowered mortality of said poultry, or in any combination thereof. The invention provides a feed additive for enhancing the poultry growth, comprising processed pomegranate parts selected from leaves, flowers, fruits, fruit cores, fruit peels, fruit pulp membranes, fruit arils, and mixtures thereof, wherein processing said parts comprises a step selected from drying, extracting, and ensiling; the process may comprise any known procedure suitable for improving the desired effects of the additive, such as pasteurization, or concentration by evaporation or ultrafiltration, etc.

The extract according to the invention may comprise at least one characteristic selected from the group consisting of punicalagins A+B in an amount of more than 2,000 mg/Kg, total phenolics determined by Folin-Ciocalteu reagent in an amount of more than 10,000 mg/Kg, and ORAC value of more than 5,000 μmol TE/100 g. In one embodiment of the invention, the additive comprises an aqueous extract of 50 Brix, containing punicalagins A+B in an amount of more than 20,000 mg/Kg, total phenolics determined by Folin-Ciocalteu test in an amount of more than 90,000 mg/Kg, and ORAC value of more than 50,000 μmol TE/100 g. The invention is directed to the use of said additive as a poultry growth promoter without growth-promoting antibiotics.

The invention relates to an enriched feed for poultry, comprising suitable known feed, the feed additive of the invention as described, the additive constituting from 0.01 to 50 kg per ton feed, usually from 0.1 to 10 kg per ton feed, and optionally at least one component selected from additional growth-promoting agent of synthetic or natural origin, other component contributing to the optimal poultry growth including probiotics, synthetic amino acids supplement, exterior enzymes improving digestibility of non-starch polysaccharides, phytase, minerals, proteins, digestible amino acids, yeast, methionine, organic or inorganic selenium, betain, chelate forms of micro elements like copper, iron, manganese and cobalt, taste-masking agent, sweetener, or tannin neutralizing component. Said enriched feed preferably lacks growth-promoting antibiotics.

The invention provides a process for manufacturing the feed additive for enhancing the poultry growth, lacking growth-promoting antibiotics, comprising, in one embodiment, mixing the pomegranate peels or parts containing them with a solvent acceptable in animal husbandry, thereby obtaining a suspension of insoluble fragments in a solution comprising pomegranate active components, separating said fragments from said solution, and removing at least a part of the solvent from the solution. In one embodiment, the solvent is completely removed, preferably in the presence of carriers, such as carbohydrates, and a powdered extract is provided. The powdered extract may comprise drying carriers, like maltodextrin, or may be mixed with other auxiliary components. In other embodiment, the process comprises milling the peels, either in dry state or in wet state, and optionally admixing other components, thereby providing homogeneous powdered or granulated solid additive.

### DETAILED DESCRIPTION OF THE INVENTION

It has now been found that adding pomegranate extract to the feed of chickens, in an amount of between 0.5 and 5 kg extract per ton of feed, may substitute for antibiotics in respect to the parameters measuring the fowl prosperity and the meat productivity.

In accordance with the invention, a part of pomegranate plant, particularly pomegranate fruit, such as pomegranate peel, or an extract thereof, may be advantageously used as a poultry growth promoter, possibly replacing classical antibiotics from the feed mixtures, particularly being added to feed as an extract, either alone or with other active or auxiliary components. Said pomegranate part or extract, added to feed, comprise at least one characteristic selected from the group consisting of punicalagins A+B in an amount of more than 2,000 mg/Kg, total phenolics determined by Folin-Ciocalteu reagent in an amount of more than 10,000 mg/Kg, and ORAC value of more than 5,000 μmol TE/100 g. Said punicalagins A+B may be in an amount of more than 20,000 mg/Kg, total phenolics determined by Folin-Ciocalteu reagent in an amount of more than 90,000 mg/Kg, and ORAC value of more than 50,000 μmol TE/100 g. Said components may include tannin-taste neutralizing component, or complexing component; a complexing component may include proteins like milk proteins. Admixed to the feed, beside the active additive, may be a taste improving additives, a sweetener, for example aspartame, a taste masking material, and the like. When omitting antibiotic growth promoter from a feed mix, the meat production need not be negatively affected if including in the mix an additive according to the invention. The additive supports poultry growth and/or enhances the feed utilization efficiency in poultry.

The invention enables to increase the productivity of the poultry industry, enhancing the poultry growth and/or the feeding efficiency, while providing safer feed and while complying with the requirements of animal welfare. The new additive will assist in ensuring the economical meat production without employing antibiotic growth promoters. The additive of the invention will be advantageously used together with other component contributing to the optimal poultry growth, including probiotics, supplementation of synthetic amino acids, exterior enzymes improving digestibility of non-starch polysaccharides, phytase, minerals, proteins, digestible amino acids, yeast, methionine, organic or inorganic selenium, betain, chelate forms of micro elements like copper, iron, manganese and cobalt, etc.

The masking agents and/or sweeteners may be added to mask astringent/bitter flavors in the feed, particularly originating from the pomegranate-based additive; said agents or sweeteners may include sodium saccharin, monoammonium glycyrrhizinate, aspartame, sucralose, thaumatin, alitame, cyclamate, neotame, potassium acesulfame, stevia extract, organic acids such as aspartic and glutamic acids, neohesperidin dihydrochalcone, etc.

In a preferred embodiment of the invention, a feed additive for poultry, aiming at substituting antibiotic growth agents, include a pomegranate-based additive combined with another component. The combinations comprising the additive according to the invention will improve the efficiency of feed utilization, namely the conversion of feed mass to the desired meat mass in poultry, for example in fattening chickens. Said combinations will enable gain in body mass, increase in feed conversion, and decrease in mortality. The invention aims at better quality of the poultry industry products, including meat, and eventually also eggs, by replacing synthetic growth promoters with natural additives. The invention aims at better results in raising poultry, including broilers, laying hens, turkeys, geese, ducks, etc., when compared to the production without any growth-promoting additives. The invention will help to adjust the poultry industry to the modern demands of the growing market, environmental protection, and animal welfare. In one embodiment of the invention, the pomegranate based additive will be added to the poultry feed with another component comprising, for example, enzymes, probiotics, and herbal extracts. In one embodiment, said pomegranate additive and said component work synergistically.

The efficiency of feed utilization in chickens of meat type is often characterized be the mass of feed in kilograms needed to produce one kilogram of meat; the efficiency, called feed conversion, is calculated as the ratio between the total (cumulative) feed consumed by one fowl and the average slaughter body weight. In the experiments carried out by the inventors, the use of antibiotics in feeding broilers lowered the value of feed conversion by between 2% and 4% in comparison with the control group without antibiotics, representing 2-4% efficiency increase in the feed utilization; the same feed conversion was achieved when replacing the antibiotics with the additive according to the invention. In view of the giant world consumption of the poultry meat, around 100 million tons a year, even a slight improvement in the feed conversion may bring huge economic benefits. Moreover, the additive of the invention may contribute also in maintaining the fowl body weight without using the antibiotic growth promoters, and further also in maintaining low mortality without said chemical growth promoters.

Instead of worldwide feeding the huge amounts of chemicals, harmless substitutes of natural origin according to the invention may help in maintaining the poultry productivity in an environmentally friendly way.

The invention will be further described and illustrated in the following examples.

### EXAMPLES

### Materials

The feed additive according to the invention was supplied to the chickens in two forms: liquid and powder. The liquid form was a pomegranate extract concentrate (PE1), 50 Brix, containing total punicalagins of 27,000 ppm, total polyphenols (determined by Folin-Ciocalteu test as gallic acid) of 92,000 ppm. The dry form (powder PE2) contained 50% pomegranate solids and 50% maltodextrin; total punicalagins of 23,000 ppm, total polyphenols (determined by Folin-Ciocalteu test as gallic acid) of 90,000 ppm.

The effects of the additives according to the invention was compared with the effects of livestock commercial antibiotics virginiamycin and avilamycin.

### Example 1

The effects of presence/absence/replacement of the antibiotics in the feed were examined. A group of 2000 Ross male broilers were randomly divided into four treatments and fed a mesh diet ad libitum. Treatment, control, did not receive a growth promoter, treatment 2 received an antibiotic growth promoter, avilamycin (Maxus™), treatment received an additive according to the invention, PE1 (comprising 50% pomegranate solids and 50% maltodextrin), 4 kg per ton feed, and treatment received an alternative plant extract. Body weight, feed intake and mortality were measured during the 42 d broiler trial. At the end of the trial, feed conversion was calculated and % cumulative mortality was recorded. Results indicated a 4.1% improved feed conversion in those birds which received an antibiotic growth promoter (Maxus™), compared to negative control (1.758 and 1.834 respectively). The addition of pomegranate extract powder improved feed conversion by 4.9% as compared to negative control treatment (1.744 and 1.834 respectively). Mortality results at end of trial were as follows: negative control 4.1%, antibiotic growth promoter 3.6% and pomegranate extract powder 1.4%.

### Example 2

The objective of this trial was to test the efficacy of a taste masking agent. The trial examined the performance parameters in 2000 Ross male broilers randomly divided into 5 treatment groups. All broilers were fed mash feed ad libitum. Treatment 1, negative control, without addition of any growth promoter in feed, treatment 2, positive control with an antibiotic growth promoter avilamycin (Maxus™), treatment 3 received 2 kg additive PE1 of the invention per ton feed, treatment 4 received 2 kg additive PE1 of the invention per ton feed with a taste masking agent including thaumatin, and treatment 5 received an alternative probiotic growth additive (lactobacillus). Use of the masking agents improved broiler body weight results from 28-44 d period by 4.2% (1480 g vs 1420 g). Final body weight results at 44 d was improved by 3.6% in those broilers that received the masking agent as compared to those without. (2748 vs 2652). Feed conversion improved by 5.4% in those broilers receiving masking agent (1.84 instead of 1.94 kg/kg). Throughout the trial the body weight of broilers receiving probiotic, lactobacillus, was consistently lower then both treatment groups receiving pomegranate extract as a feed supplement.

### Example 3

The objective of this trial was to determine the efficacy and dosage of the liquid form of pomegranate extract (alternative growth promoter) as compared to powder form. Trial set-up was similar to experiment number 1. A group of 2000 Ross male broilers was divided randomly into 5 treatment groups. Treatment 1 received an antibiotic growth promoter, treatments 2 through 5 received pomegranate extract in either liquid or powder form in different dosage levels (0.5 kg/ton and 1.5 kg/ton feed). All pomegranate extract treatment groups contained the flavor masking agent (as in Example 2). Body weight results were improved in broilers receiving pomegranate extract as compared to antibiotic growth promoter treatment (2620 g-2710 g versus 2520 g). All broilers in all the treatments receiving pomegranate extract had an improved feed conversion (1.84-1.91 versus 1.96).

### Example 4

Two groups of broilers (Heritage) were placed in two commercial temperature-controlled houses. Each house contained approximately 24000 broilers at a density of 13.5 broilers/m². The broilers were fed by ad libitum pellet feed from day 1 till marketing at day 42. The 1^{st} group got the antibiotic growth promoter virginiamycin, and the 2^{nd} group got additive according to the invention PE1, in liquid form, 2.5 kg per ton feed. Results at marketing showed that broilers receiving PE1 had improved body weight (2.8%), better feed conversion (1.1%), and lower mortality (8%) then the control group which received the antibiotic growth promoter.

While this invention has been described in terms of some specific examples, many modifications and variations are possible. It is therefore understood that within the scope of the appended claims, the invention may be realized otherwise than as specifically described.

1. A method for enhancing the growth of poultry, comprising admixing to feed for said poultry an additive derived from pomegranate plant.

2. A method according to claim 1, wherein said additive is prepared by processing a pomegranate part selected from the group consisting of leaves, flowers, fruits, fruit cores, fruit peels, fruit pulp membranes, fruit arils, and mixtures thereof.

3. A method according to claim 1, wherein said additive is a dried pomegranate part, milled pomegranate part, an extract of a pomegranate part or a concentrate thereof, or a mixture thereof.

4. A method according to claim 1 for enhancing the growth of poultry, comprising adding to the feed for said poultry an additive comprising pomegranate peels selected from the group consisting of dried pomegranate peels, an extract from pomegranate peels, and silage of pomegranate peels.

5. A method according to claim 1, wherein said additive is added to said feed in an amount of from 0.01 kg to 50 kg per one ton feed.

6. A method according to claim 1, wherein said additive further comprises at least one component selected from the group consisting of growth-promoting agent of synthetic or natural origin, probiotic, prebiotic, methionine, synthetic amino acids supplement, digestible amino acids, exterior enzymes improving digestibility of non-starch polysaccharides, phytase, proteins, betain, yeast, organic or inorganic material comprising selenium, minerals, chelate forms of micro elements like copper, iron, manganese and cobalt, taste-masking agent, sweetener, and tannin neutralizing component.

7. A method according to claim 1, wherein said additive is a solid or a liquid further comprising a component selected from taste mask, sweetener, and tannin neutralizing component.

8. A method according to claim 1, wherein said growth enhancement results in increased average weight of said poultry.

9. A method according to claim 1, wherein said growth enhancement results in increased efficiency of the feed utilization.

10. A method according to claim 1, wherein said 1, wherein said growth enhancement results in lowered mortality of said poultry.

11. A method according to claim 1, wherein said additive substitutes for growth-promoting antibiotics, and wherein said poultry comprises chickens and turkeys.

12. A method according to claim 1, wherein said poultry comprises chickens for fattening.

13. A method for enhancing the growth of poultry according to claim 1, comprising omitting growth-promoting antibiotics in said feed.

14. A feed additive for enhancing poultry growth, comprising processed pomegranate parts selected from leaves, flowers, fruits, fruit cores, fruit peels, fruit pulp membranes, fruit arils, and mixtures thereof, wherein processing said parts comprises a step selected from drying, extracting, and ensiling.

15. A feed additive according to claim 14, wherein said extract comprises at least one characteristic selected from the group consisting of punicalagins A+B in an amount of more than 20,000 mg/kg, total phenolics determined by Folin-Ciocalteu reagent in an amount of more than 90,000 mg/kg, and ORAC value of more than 50,000 μmol TE/100 g.

16. A feed additive according to claims 14 for use as a poultry growth promoter without growth-promoting antibiotics.

17. A feed additive according to claims 14, further comprising at least one component selected from the group consisting of additional growth-promoting agent of synthetic or natural origin, probiotic, prebiotic, methionine, synthetic amino acids supplement, digestible amino acids, exterior enzymes improving digestibility of non-starch polysaccharides, phytase, proteins, betain, yeast, organic or inorganic material comprising selenium, minerals, chelate forms of micro elements like copper, iron, manganese and cobalt, taste-masking agent, sweetener, and tannin neutralizing component.

18. An enriched feed for poultry, comprising poultry feed and the feed additive of claim 14, the additive constituting from 0.01 to 50 kg per 1 ton feed.

19. An enriched feed for poultry without growth-promoting antibiotics, comprising poultry feed and the feed additive of claim 14.

20. A process for manufacturing the feed additive of claim 14 for enhancing poultry growth, lacking growth-promoting antibiotics, comprising mixing the pomegranate peels or parts containing them with a solvent acceptable in animal husbandry, thereby obtaining a suspension of insoluble fragments in a solution, and removing at least a part of said solvent from said solution.
